(19) United States
(12) Patent Application Publication
(10) Pub. No.: US 2005/0222102 A1
(43) Pub. Date: Oct. 6, 2005

(54) Treatment of rhinitis with anticholinergics alone or in combination with antihistamines, phosphodiesterase 4 inhibitors, or corticosteroids

(75) Inventors: Joachim Maus, Muhlheim (DE); Ursula Petzold, Bickenbach (DE); Istvan Szelenyi, Schwaig (DE); Torsten Hoffmann, Radebeul (DE); Mario Weingart, Dresden (DE)

Correspondence Address: VENABLE LLP, P.O. BOX 34385, WASHINGTON, DC, 20045-9998, US

(73) Assignee: Sofotec GmbH & Co. KG, Frankfurt am Main (DE)

(21) Appl. No.: 11/051,470

(22) Filed: Feb. 7, 2005

Related U.S. Application Data
(60) Provisional application No. 60/541,950, filed on Feb. 6, 2004.

(51) Int. Cl. A61K 31/573; A61K 31/55; A61K 31/519; A61K 31/401

(52) U.S. Cl. 514/171; 514/423; 514/252.16; 514/262.1; 514/217.05

(57) ABSTRACT

The present invention provides novel combinations comprising a topical anticholinergic drug alone or in combination with topically administered antihistamines, topically or orally administered phosphodiesterase 4 inhibitors or topical corticosteroids for the treatment of rhinitis of various origins. It further comprises presentation of these combinations in locally applied formulations and includes various pharmaceutical formulations suitable for topical application, e.g. nasal sprays, nasal drops, emulsions, pastes, creams and gels.

The present invention provides novel combinations comprising a topical anticholinergic drug alone or in combination with topically administered antihistamines, topically or orally administered phosphodiesterase 4 inhibitors or topical corticosteroids for the treatment of rhinitis of various origins. It further comprises presentation of these combinations in locally applied formulations and includes various pharmaceutical formulations suitable for topical application, e.g. nasal sprays, nasal drops, emulsions, pastes, creams and gels.

Rhinitis is a global health concern and shares a high comorbidity with asthma. It is a complex disease affecting approximately 20% of the population. Rhinitis occurs in different types: allergic or atopic rhinitis including seasonal and perennial forms. Both seasonal and perennial allergic rhinitis are triggered by indoor or outdoor allergens. The mechanism of perennial rhinitis with non-allergic triggers is not well understood. It is an allergy-like condition but not triggered by allergens. Idiopathic non-allergic rhinitis or vasomotor rhinitis is characterized by nasal congestion and postnasal drip in response to temperature and humidity changes, smoke, odors, and emotional upsets. In general, rhinitis is defined as inflammation of the nasal membranes and is characterized by a symptom complex that consists of any combination of the following: sneezing, nasal congestion, nasal itching, and rhinorrhea. Clinical symptoms of seasonal allergic rhinitis typically include nasal itching and irritation, sneezing and watery rhinorrhea, frequently accompanied by nasal congestion. The perennial allergic rhinitis clinical symptoms are similar, except that nasal blockage may be more pronounced. Either type of allergic rhinitis may also cause other symptoms such as itching of the throat and/or eyes, epiphora and oedema around the eyes. These symptoms may vary in intensity from the nuisance level to debilitating. Other types of rhinitis present the same types of symptoms. Failure of treatment of rhinitis may lead to other disorders including infection of the sinuses, ears and lower respiratory tract. While rhinitis itself is not life threatening (unless accompanied by severe asthma or anaphylaxis), morbidity from the condition can be significant. Allergic rhinitis often coexists with other disorders, such as asthma, sinusitis, nasal polyps, allergic conjunctivitis, and atopic dermatitis. Rhinitis may also contribute to learning difficulties, sleep disorders, drowsiness and fatigue. All these symptoms can frequently lead to significant impairment of quality of life. As related to a patient's quality of life, rhinorrhea is reported as the most prominent and distressing symptom of allergic rhinitis.

Recent research suggests that different allergic diseases, such as rhinitis, asthma, allergic conjunctivitis and chronic idiopathic urticaria, are evoked by common pathological mechanisms characterised by the release of histamine and other inflammatory mediators.

Histamine is an important mediator released from cells that line the walls of the nasal mucous membranes (mast cells). When released, histamine is known to bind competitively to local histamine H₁-receptors and cause sneezing, nasal itching, and swelling of the nasal membranes. The primary action of antihistamines relates to their ability to bind competitively to H₁-histamine receptors on target organ sites, thereby blocking the ability of histamine to bind to these receptors. These so-called first-generation antihistamines such as brompheniramine, chlorpheniramine, diphenhydramine, promethazine, and hydroxyzine have lipophilic chemical properties, which contribute to both their sedating and their anticholinergic effects.

The sedating side effects of antihistamines have stimulated the development and marketing of the so-called 2nd generation antihistamines such as loratadine, cetirizine, terfenadine, astemizole, azelastine, levocabastine, fexofenadine, mizolastine, etc. All are less lipophilic than first-generation antihistamines, conferring a reduction in their ability to cross the blood-brain barrier and thereby cause sedation. However, some of these second-generation antihistamines have a concomitant diminution of anticholinergic effects and decreased potency for controlling rhinorrhea. Therefore, if a neurologic mechanism or predominantly rhinorrhea symptoms are present, an anticholinergic might be the treatment of choice.

Today a 3rd generation of antihistamines is under discussion. Desloratadine and levocetirizine which are either metabolites or isomers of 2nd generation antihistamines are considered to fulfill the 3rd generation criteria. Their advantage compared to 2nd generation products is seen in an improved safety profile (e.g. no interference with cardiac conduction). Desloratadine and levocetirizine are free of antimuscarinic/anticholinergic effects.

There are three topical (nasal) histamine H₁-receptor antagonists, azelastine, levocabastine, and dimetinden available which are well established as anti-rhinitis therapy. Azelastine is a pharmacologically distinct histamine H₁-receptor antagonist with a broad spectrum of antiallergic and anti-inflammatory activity (Szelenyi et al., Agents Actions. 1991; 34(Suppl):295-311). Azelastine has established antiallergic and anti-inflammatory effects that are unrelated to H₁-receptor antagonism, including inhibitory effects on the synthesis of leukotrienes, kinins, and cytokines; the generation of superoxide free radicals; and the expression of the intercellular adhesion molecule 1 (ICAM-1) (Schmidt et al., J Lipid Mediat 1992; 5:13-22, Kusters et al., Arzneimittelforschung 2002; 52:97-102). Levocabastine is a highly potent and specific histamine H₁-receptor antagonist which has been developed for topical application as eyedrops and nasal spray. Results of comparative clinical trials suggest that topical levocabastine is at least as effective as oral antihistamines for the treatment of rhinitis, and it is suggested as an attractive alternative to oral antihistamines as first-line therapeutic option (Janssens and Van den Bussche, Clin Exp Allergy 1991; 21(Suppl 2):29-36, Knight, Br J Clin Pract 1994; 48:139-43, Yanez and Rodrigo, Ann Allergy Asthma Immunol 2002; 89:479-84). Azelastine and levocabastine are available worldwide as nasal spray formulations and approved for treatment of allergic rhinitis; in the United States azelastine is also available to treat non-allergic vasomotor rhinitis.

Histamine H₁-receptor antagonists have been proven efficacious for preventing and relieving sneezing, itching, and other symptoms of the early allergic response, but have not been found to be very effective for relief of the nasal congestion which is a typical characteristic of the later stages of an allergic reaction (Pien, Cleve Clin J Med 2000; 67:372-80, Salmun, Expert Opin Investig Drugs 2002; 11:259-73).

The release of histamine is an important mechanism underlying some of the symptoms of rhinitis. The symptom of rhinorrhea, however, is largely attributable to a neuronal mechanism; specifically, to the effects of acetylcholine on nasal cholinergic receptors, rather than to the action of histamine. This can be demonstrated by observing that histamine challenge on one side of the nose produces an increase in nasal secretions, on the other side as well. The reflex increase in secretions on the non-challenged side can be inhibited by pre-medication with an anticholinergic agent, i.e. an agent which acts by blocking the action of acetylcholine or cholinergic receptors.

Anticholinergic agents are exemplified by the belladonna alkaloids atropine and scopolamine, which inhibit the muscarinic action of acetylcholine on structure innervated by postganglionic cholinergic nerves. These agents typically inhibit the nasal secretory mechanism and cause drying of the nasal membranes. However, intranasal anticholinergics do not alter physiologic nasal functions (e.g., sense of smell, ciliary beat frequency, mucociliary clearance, or the air conditioning capacity of the nose). Anticholinergic agents also are known to exert central effects which include pupil dilatation and stimulation and/or depression of the central nervous system.

Antimuscarinic treatment of rhinitis has a relatively long history leading to its present day use as an effective antisecretory drug for watery rhinorrhea. Watery rhinorrhea is, in fact, a common problem in some individuals with rhinitis. Some of these secretions come from parasympathetic stimulation of the many mucus and serous glands in the nasal mucosa, therefore a local (nasal) anti-cholinergic may be advantageous. Novel anticholinergic pharmaceuticals have been developed which have a limited capacity to pass across the blood-brain barrier, and therefore have a limited capacity to produce central effects. Examples of these agents are the quaternary ammonium compounds methscopolamine, ipratropium, oxitropium, tiotropium and the enantiomers of glycopyrrolate. Present formulations are, however, limited to ipratropium bromide (Witek, Respir Care Clin N Am 1999; 5:521-36). Ipratropium is a safe and effective therapy for control of rhinorrhea in patients with rhinitis (Meltzer et al., Ann Allergy Asthma Immunol 1997; 78:485-91, Dockhorn et al., Ann Allergy Asthma Immunol 1999; 82:349-59). There was an improvement in patient quality of life, as well as a substantial reduction in the need for other medications (antihistamines, decongestants, and nasal steroids) used to treat perennial rhinitis symptoms (Druce et al., Ann Allergy 1992; 69:53-60, Grossman et al., J Allergy Clin Immunol 1995; 95:1123-7, Kaiser et al., Allergy Asthma Proc 1998; 19:23-9). There was no rebound increase in rhinorrhea following discontinuation of the ipratropium administration (Kaiser et al., Allergy Asthma Proc 1998; 19:23-9). Ipratropium, like all other quarternary ammonium derivatives, is poorly absorbed by the nasal mucosa. Therefore, its use is not associated with adverse systemic effects. Local adverse effects (eg, dryness, epistaxis, irritation) may occur.

Recently, it has been demonstrated that patients with symptomatic non-allergic rhinitis or even asymptomatic patients with allergic rhinitis out of pollen season present a nasal hyperreactivity to methacholine which could be prevented by ipratropium (Marquez et al., Am J Rhinol 2000; 14:251-6). Ipratropium is effective in controlling rhinorrhea and shows a good effect on nasal congestion (Milgrom et al., Ann Allergy Asthma Immunol 1999; 83:105-11). Additionally it is safe and increases the ability of the nose to condition cold, dry air (Assanasen et al., Am J Respir Crit Care Med 2000; 162:1031-7).

Allergic rhinitis involves inflammation of the mucous membranes of the nose, eyes, eustachian tubes, middle ear, sinuses, and pharynx. The nose invariably is involved, and the other organs are affected in certain individuals. Inflammation of the mucous membranes is characterized by a complex interaction of inflammatory mediators. Consequently, one of the most effective therapies of rhinitis is an anti-inflammatory medication. Because of their efficacy, nasal corticosteroids remain the cornerstone in the treatment of rhinitis. Despite the long history and the documented efficacy of these drugs in controlling rhinitis, concerns still abound regarding the safety of these drugs in children, most specifically related to the potential for adrenal suppression and growth retardation. Recently published studies suggest that adrenal function remains intact when low and moderate doses of these drugs are used. Long-term studies of growth in children suggest that despite an initial decrease in growth velocity, ultimate adult height is not affected significantly by the use of nasal corticosteroids (Bazzy-Asaad, Curr Opin Pediatr 2001; 13:523-7, Allen, Pediatrics 2002; 109(2 Suppl):373-80, Skoner, Curr Opin Pulm Med 2002; 8:45-9). Since intranasally applied corticosteroids may circulate systemically, a risk of growth suppression in children treated with these drugs cannot be ruled out. Therefore, there is still need to improve the present therapy with corticosteoids by using combination with other medications in order to improve the safety by decreasing the steroid dose.

The increasing prevalence of allergic rhinitis, its impact on individual quality of life and social costs, as well as its role as a risk factor for asthma, underline the need for improved treatment options for this disorder. Phosphodiesterase 4 (PDE4) is a major cyclic adenosine-3′,5′-monophosphate-metabolizing enzyme in immune and inflammatory cells, airway smooth muscle, and pulmonary nerves. Selective inhibitors of this enzyme have been shown a broad spectrum of activity in experimental models of rhinitis (Marx et al., J Allergy Clin Immunol 1997; 99:S444, Poppe et al., Allergy 2000; 55(Suppl. 63):270). An increased activity of PDE4 has been observed in peripheral leukocytes from patients suffering from rhinitis. Rolipram, one of the first selective PDE4 inhibitors, effectively suppressed this phenomenon indicating that the use of a specific and well tolerable PDE4 inhibitor may be effective in the treatment of rhinitis (Raderer et al., Wien Med Wochenschr. 1995; 145:456-8, Baraniuk and Tai, Curr Allergy Asthma Rep 2002; 2:191-2). Recently, it has been reported that the novel PDE4 inhibitor, roflumilast, effectively controls symptoms of allergic rhinitis (Schmidt et al., J Allergy Clin Immunol 2001; 108:530-6). Thus PDE4 inhibitors may be a future treatment option in rhinitis, as well. The class-associated side effects, mainly nausea and emesis, appear to have been at least partially overcome by the topical (nasal or inhaled) administration as demonstrated by AWD 12-281.

### DESCRIPTION OF THE INVENTION

Racemic glycopyrrolate has four diastereoisomers. Although the diastereoisomers are nonselective muscarinic receptor antagonists, one of its isomers, the R,R-enantiomer shows a kinetic selectivity for muscarinic M₃ receptors. Because of the quaternary nature, it is poorly absorbed when swallowed and penetrates neither placental nor blood-brain barriers. Similarly, its oral absorption is slow and erratic. A further advantage of the drug is that it is excreted mainly as unchanged drug renally (Ali-Melkkila et al. Acta Anaesthesiol Scand 1993; 37:633-42). Racemic glycopyrrolate given as an aerosol does provide long lasting bronchodilatation from its blocking action on smooth muscle (Tzelepis et al., Eur Respir J 1996; 9:100-3).

Intranasal anticholinergic agents such as ipratropium, tiotropium, and glycopyrrolate could be used for reducing rhinorrhea (“watery secretion”) in patients with allergic or vasomotor rhinitis. These drugs may be basically used alone or in combination with other medications.

In the clinical practice, histamine H₁-receptor antagonists, decongestants, corticosteroids and anticholinergics are most commonly used pharmacological agents for the treatment of rhinitis. Due to the complexity of symptoms, combinations of different drugs are often indicated. For example, it has been common to concurrently administer sympathomimetic decongestant drugs, such as phenylpropanolamine, pseudoephedrine, xylometazoline, oxymetazoline, etc. orally or intranasally. Although several orally applied combination products containing both histamine H₁-receptor antagonists and decongestants are now commercially available, not all allergy sufferers should use these decongestants drugs, due to their frequently observed topical, central nervous system and cardiovascular side effects which include rhinorrhea, agitation, sleeplessness, tachycardia, angina pectoris, and hypertension. Moreover, topical vasoconstrictors may also be added to the antihistamines for temporary relief but their use should be limited to less than 5 days to minimize the risk of developing rebound nasal congestion. These observations emphasized the need for new anti-allergic agents with a broader spectrum of activity and an improved safety profile.

The 2nd and 3rd generation antihistamines are frequently prescribed in preference to the 1st generation antihistamines in order to avoid sedation, despite their lack of anticholinergic effect. The formulation of an anticholinergic agent together with a non-sedating antihistamine would “reinstate” the anticholinergic effects which have been lost in the transition from first-generation to second-generation antihistamines. It is, therefore, an object of the present invention to devise nasal antihistaminic formulations that are non-sedating, but which still confer the anticholinergic properties forfeited by the new non-sedating antihistamines.

Unrelated to their function of binding to H₁ histamine receptors, the 1st generation antihistamines produce sedation, an unwanted side effect, but also provide anticholinergic effects, which are helpful for reducing secretions and controlling rhinorrhea. The 2nd generation antihistamines, which are relatively nonsedating, have been developed but are lacking in anticholinergic efficacy. Despite the abundance of presently marketed formulations for addressing the symptoms for allergic rhinitis, no medicinal formulation is presently available which provides both antihistaminic and anticholinergic actions in an essentially nonsedating manner.

The combination of a topical administered ipratropium and an oral administered terfenadine is known from Finn et al. (Am J Rhinol 1998; 12:441-9). However, the combinative administration of an oral drug with a nasal spray can hardly be realized in the daily practice. More convenient would be a therapy with a combination consisting of two topically applied drugs, for example, R,R-glycopyrrolate with azelastine or levocabastine. This has also to be considered from an economical point of view in order to reduce daily therapy costs.

Similar, less convenient combinative therapies have also been described for topical corticosteroid in combination with oral antihistamines. Undoubtedly, topical corticosteroids are highly effective drugs in allergic rhinitis. However, the onset of their anti-rhinitis action takes a longer time, usually, some days. To achieve an acute improvement, topical antihistamines or decongestants can be administered. The topical (nasal) combination consisting of an anticholinergic drug (e.g. ipratropium, tiotropium, glycopyrrolate, especially, R,R-glycopyrrolate) and a corticosteroid (e.g. beclomethasone, budesonide, ciclesonide, fluticasone, mometasone, triamcinolone, loteprednol) may be more effective and safe in the treatment of rhinitis in patients with predominantly rhinorrhoea symptoms. As the dose of a steroid could be reduced when it is combined with an anticholinergic agent, it can be expected that the risk to induce undesired steroid-effects is also minimized.

In addition to well established pharmacological therapies with antihistamines, corticosteroids, decongestants and mast cell stabilizers, new therapeutic options become increasingly important. As already mentioned, PDE4 inhibitors represent a novel therapeutically promising class of drugs which may be effective in the treatment of rhinitis, as well. Unfortunately, the effects of prototype PDE4 inhibitors have been compromised by side effects such as nausea and emesis and the clinical use of those compounds is still limited. AWD 12-281 represents a novel class of PDE4 inhibitors. In animal studies, it was devoid of emesis and signs of nausea up to high oral doses. AWD 12-281 was highly effective in different animal models of asthma and rhinitis. Its combination with an anticholinergic agent such as glycopyrrolate, especially R,R-glycopyrrolate could considerably increase its therapeutic effectiveness.

There is now surprising experimental evidence that glycopyrrolate, especially, the R,R-isomer causes a longer-lasting reduction in the watery secretion in experimental allergic rhinitis models than typical for anticholinergic agents and with lower side-effects than expected

The present invention describes the surprising effect that topically applied anticholinergics such as glycopyrrolate, its enantiomers, especially R,R-glycopyrrolate or diastereoisomers or physiologically acceptable salts administered alone or in combination with topically (nasal) applied antihistamines (histamine H₁-receptor antagonists), phosphodiesterase 4 inhibitors or corticosteroids or their physiologically acceptable salts are effective and safe in the treatment of rhinitis. Glycopyrrolate belongs to the so-called anticholinergic drugs and antagonizes the neurotransmitter acetylcholine at its receptor site. This effect leads to a considerably reduced watery secretion in rhinitis. Topically administered (nasal) antihistamines such as levocabastine, azelastine, and dimetinden antagonize histamine at the histamine H₁-receptor resulting in attenuation of several symptoms of rhinitis. Based on pre-clinical data Phosphodiesterase 4 inhibitors are also effective in the treatment of rhinitis. Topically (intranasal) applied corticosteroids have become the mainstay of therapy in rhinitis. However, they given alone are often less active in suppression of nasal congestion and rhinorrhea, respectively. The anticholinergic glycopyrrolate is especially suitable for the treatment of rhinitis characterized by an increased watery secretion. The combination disclosed in the present invention of glycopyrrolate with an antihistamine, a phosphodiesterase 4 inhibitor or a corticosteroid formulated as a nasal spray shows an overadditive effect compared to the monocompounds alone.

All combinative drugs mentioned before have similar pharmacokinetic behaviors. All they have long-lasting effects. Therefore, a frequent use of the combinations is not necessary. Consequently, the combination of such drugs leads to a better efficacy and an improved tolerability.

Anticholinergic Agents Plus Antihistamines

The special combination therapy disclosed in this invention comprises administering locally racemic glycopyrrolate, one of its enantiomers, especially R,R-glycopyrrolate or a mixture thereof, with intranasal azelastine, levocabastine or dimetinden. The compounds can be administered simultaneously or sequentially or in a fixed combination. They may be given together in a single dosage form. Or they may be administered as two different formulations which may be the same or different. They may be given at the same time (simultaneously) or they can be administered either close in time or remotely, such as where the anticholinergic R,R-glycopyrrolate is given in the evening and the antihistamine azelastine or levocabastine or dimentiden is given in the morning.

The active ingredient may be given from 1 to 3 times a day, sufficient to exhibit the desired activity. Preferably, the active components are given about once a day, more preferably twice a day.

As for the amount of drug administered, R,R-glycopyrrolate can be administered intrariasally in an amount of 5 to 500 μg/day in adult humans with the preference of 15 to 300 μg/day in dependence of the magnitude of rhinorrhea. A dosage range between 5 and 100 μg/day is especially preferred. Azelastine-HCI can be administered intranasally in conformity with approved labeling in an amount of 140 to 1.120 μg/day with the preference between 280 and 560 μg/day.

Anticholiergic Agents Plus Corticosteroids

The special combination therapy disclosed in this invention comprises administering locally racemic glycopyrrolate, one of its enantiomers, especially R,R-glycopyrrolate or a mixture thereof with an intranasal corticosteroid, preferably budesonide or ciclesonide or fluticasone, beclomethasone, mometasone, flunisolide or loteprednol. The compounds. can be administered simultaneously or sequentially or in a fixed combination. They may be given together in a single dosage form. Or they may be administered as two different formulations which may be the same or different. They may be given at the same time (simultaneously) or they can be administered either close in time or remotely, such as where the anticholinergic R,R-glycopyrrolate is given in the evening and the corticosteroid is given in the morning. Formulations are within the skill of the art.

The active ingredient may be given from 1 to 3 times a day, sufficient to exhibit the desired activity. Preferably, the active components are given about twice a day, more preferably once a day.

As for the amount of drug administered, R,R-glycopyrrolate can be administered in an amount of 5 and 500 μg/day adult human with the preference of 15 and 300 μg/day in dependence of the magnitude of rhinorrhea. A dosage range between 5 and 100 μg/day is especially preferred. Corticosteroids (budesonide or ciclesonide or fluticasone or mometasone or beclometasone or flunisolide or loteprednol) can be administered in conformity with approved labeling in an amount of 100 to 800 μg/day with the preference between 200 and 400 μg/day.

Anticholinergic Agents Plus Phosphodiesterase 4 Inhibitors

The special combination therapy disclosed in this invention comprises administering locally racemic glycopyrrolate, one of its enantiomers, especially R,R-glycopyrrolate or a mixture thereof with a intranasal PDE4 inhibitor, for example, AWD 12-281 or an oral PDE4 inhibitor, for example roflumilast. The compounds can be administered simultaneously or sequentially or in a fixed combination. They may be given together in a single dosage form. Or they may be administered as two different formulations which may be the same or different. They may be given at the same time (simultaneously) or they can be administered either close in time or remotely, such as where the anticholinergic R,R-glycopyrrolate is given in the evening and the PDE4 inhibitor AWD 12-281 is given in the morning.

The active ingredient may be given from 1 to 3 times a day, sufficient to exhibit the desired activity. Preferably, the active components are given about once a day, more preferably twice a day.

As for the amount of drug administered, R,R-glycopyrrolate can be administered in an amount of 5 and 500 μg/day adult human with the preference of 15 and 300 μg/day in dependence of the magnitude of rhinorrhea. A dosage range between 5 and 100 μg/day is especially preferred. The PDE4 inhibitor AWD 12-281 can be administered in an amount of 200 to 2.000 μg/day with the preference between 400 and 1.000 μg/day.

The effects mentioned above are observed both when the two active substances are administered simultaneously in a single active substance formulation and when they are administered successively in separate formulations. According to the invention, it is preferable if the two active substance ingredients are administered simultaneously in a single formulation.

Various pharmaceutical formulations, e.g. nasal sprays or nasal drops, are suitable for topical (intranasal) application. The dosage forms may also include an emulsion, a paste, a cream and/or a gel. These dosage forms are part of the present invention.

If the water solubility of the drug substance sufficient like in the case of azelastine hydrochloride, formulations containing such active compound can preferably be formulated as solutions. Active compounds which are virtually water-insoluble like glycopyrrolate for example are therefore formulated as an aqueous suspension. In a formulation in which the active compounds are combined the active compounds could be present both dissolved in water, one active compound dissolved in water and the other suspended in water or both active compounds suspended in water depending on the water solubility of the drug substances.

In addition to the active compounds the pharmaceutical preparations according to the invention can contain further constituents such as preservatives, stabilizers, isotonicizing agents, thickeners, suspension stabilizers, excipients for pH adjustment, buffer systems, wetting agents and others, e.g. colorants.

Antimicrobial preservative substances include, for example: benzalkonium chloride, chlorobutanol, thiomersal, methylparaben, propylparabe, sorbic acid and it salts, sodium edetate, phenylethyl alcohol, chlorhexidine hydrochloride and bromide, chorhexidine acetate, chlorhexidine digluconate, chlorocresol, phenymercury salts, phenoxyethanol, cetylpyridinium chloride or bromide.

A combination of sodium edetate and benzalconium chloride can be suitable used as a preservative. Sodium edetate is used in concentrations of 0.05 to 0.1%, and benzalkonium chloride in concentrations of 0.005 to 0.05% wt., based on the composition.

Suitable excipients for the adjustment of the isotonicity or osmolarity of the formulations are, for example: sodium chloride, potassium chloride, mannitol, glucose, sorbitol, glycerol, propylene glycol. In general, these excipients are employed in concentrations from 0.1 to 10%.

The formulations of the invention can also include suitable buffer systems or other excipients for pH adjustment in order to establish and maintain a pH of the order of magnitude of 4 to 8, preferably of 5 to 7.5. Suitable buffer systems are citrate, phosphate, tromethamol, glycine, borate, acetate. These buffer systems can be prepared from substances such as, citric acid, monsodium phosphate, disodium phosphate, glycine, boric acid, sodium tetraborate, acetic acid, sodium acetate. Further excipients can also be used for pH adjustment, such as hydrochloric acid or sodium hydroxide.

In order to prepare a stable aqueous suspension containing a water-insoluble active compound, suitable suspension stabilizers and suitable wetting agents are furthermore necessary in order to disperse and to stabilize the suspended drug substance in a suitable manner.

Suitable suspension stabilizers are water-soluble or partly water-soluble polymers: these include, for example, methylcellulose (MC), sodium carboxymethylcellulose (Na-CMC), hydroxypropylmethylcellulose (HPMC), polyvinyl alcohol (PVAL), polyvinylpyrrolidone (PVP), polyacrylic acid, polyacrylamide, gellan gum (Gelrite®), hydrated alumina (Unemul®), dextrins, cyclodextrins, cellulose acetate phtalate, and mixtures of microcristalline cellulose (different types of Avicel®) and sodium carboxymethylcellulose. These substances can simultaneously serve as thickeners in order to increase the viscosity and thereby to prolong the contact of the drug substances with the tissue at the application site.

Suitable wetting agents are, for example: benzalkonium chloride, cetylpyridinum chloride, tyloxapol, various polysorbates (Tween®), and further polyethoxylated substances and poloxamers.

For nasal administration of solutions or suspensions according to the invention, various devices are available in the art for the generation of drops, droplets and sprays. For example, formulations can be administrated into the nasal passages by means of a dropper (or pipet) that includes a glass, plastic or metal dispensing tube. Fine droplets and sprays can be provided by an intranasal pump dispenser or squeeze bottle as well known to the art.

The invention also includes a kit containing one or more unit dehydrated doses of one or more drug substances as well as any required excipients of the formulation, ready for preparation of a solution or suspension by addition of a suitable amount of sterile or nonsterile water.

The following examples describe the invention without limiting it.

### EXAMPLE 1

### Nasal Spray or Nasal Drops Containing Azelastine Hydrochloride (0.1 %)

| | |
|---|---|
| Azelastine hydrochloride | 0.1000 g |
| Hydroxypropylmethylcellulose | 0.1000 g |
| Sodium edetate | 0.0500 g |
| Sodium hydroxide | q.s. pH 6.0 |
| Sorbitol solution 70% | 6.6666 g |
| Purified water | to 100 ml |

Preparation of the Solution:

Introduce about 45 kg of purified water into a suitable stirrer-equipped container. Add the azelastine hydrochloride, hydroxypropylmethylcellulose, sodium edetate, and sorbitol solution successively thereto and dissolve with stirring. Make up the resulting solution to a volume of 49.5 liters with purified water. Adjust the pH of the solution to pH 6.0 using 1 N sodium hydroxide solution. Make up to the final volume of 50.0 liters using purified water and stir. Filter the solution through a membrane filter having a pore size of 0.2 μm and dispense into bottles.

### EXAMPLE 2

### Nasal Spray or Nasal Drops Containing Azelastine Hydrochloride and Glycopyrrolate

| | |
|---|---|
| Azelastine hydrochloride | 0.1000 g |
| R,R-Glycopyrrolate | 0.055 g |
| Hydroxypropylmethylcellulose | 0.1000 g |
| Sodium edetate | 0.0500 g |
| Benzalkonium chloride | 0.0125 g |
| Sorbitol solution 70% | 6.600 g |
| Purified water | to 100 ml |

Preparation of the Solution:

Introduce 80% of purified water into a suitable stirrer-equipped container. Add the Azelastine-HCI, Glycopyrrolate, Hydroxypropylmethylcellulose, Benzalkonium chloride, Sodium edetate and Sorbitol solution successively thereto and dissolve with stirring. Make up to the final volume using purified water and stir. Filter the solution through a membrane filter having a pore size of 0.2 μm and dispense into bottles.

1. A combination of topical anticholinergics with antihistamines, phosphodiesterase (PDE) 4 inhibitors or corticosteroids or their physiologically acceptable salts for the treatment of allergic seasonal and perennial rhinitis.

2. (canceled)

3. (canceled)

4. The combination according to claim 1 wherein the anticholinergic is selected from the group consisting of glycopyrrolate, an enantiomer thereof, a diastereoisomer thereof, a physiologically acceptable salt thereof and mixtures thereof.

5. The combination according to claim 4 wherein the anticholinergic is R,R-glycopyrrolate or a physiologically acceptable salt thereof.

6. The combination according to claim 1 wherein the phosphodiesterase (PDE) 4 inhibitors are selected from the group consisting of Roflumilast, AWD-12-281, and their physiologically acceptable salts.

7. The combination according to claim 1 wherein the antihistamines are selected from the group consisting of azelastine, levocarbastine, dimentiden and mometasone and their physiologically acceptable salts.

8. The combination according to claim 1 wherein the corticosteroids are selected from the group consisting of budesonide, ciclesonide, fluticasone, beclometasone, mometasone, flunisolide and loteprednol and their physiologically acceptable salts.

9. A pharmaceutical for the treatment of allergic or non allergic rhinitis or rhinorrhea associated with common cold comprising at least a topical anticholinergic and at least an inhibitor of phosphodiesterase type 4 or an antihistamine or a corticosteroid or their physiologically acceptable salts.

10. The pharmaceutical according to claim 9 wherein the anticholinergic is selected from the group consisting of glycopyrrolate, an enantiomer thereof, a diastereoisomer thereof, a physiologically acceptable salt thereof and mixtures thereof.

11. The pharmaceutical according to claim 10 wherein the anticholinergic is R,R-glycopyrrolate or a physiologically acceptable salt thereof.

12. The pharmaceutical according to claim 9 wherein the anticholinergic, phosphodiesterase inhibitor, corticosteroid and/or physiologically acceptable salt(s) are presented in fixed or free combination for simultaneous, sequential or separate administration.

13. The pharmaceutical according to claim 9 that additionally contains at least one excipient, adjunct, or additive in a pharmaceutical form suitable for topical application.

14. The pharmaceutical according to claim 9, in the form of a nasal spray or nasal drops or an emulsion or a paste or a cream or a gel.

15. (canceled)

16. (canceled)

17. (canceled)

18. (canceled)

19. (canceled)

20. A method of treating allergic or non allergic rhinitis or rhinorrhea associated with common cold in an individual in need thereof, comprising administering an effective amount of the pharmaceutical according to claim 9, wherein the anticholinergic is presented in a daily dose between 5 and 500 μg/day.

21. The method according to claim 20, wherein the anticholinergic is presented in a daily dose between 15 and 300 μg/day.

22. The method according to claim 21 wherein the anticholinergic is presented in a daily dose between 5 and 100 μg/day.

23. The method according to claim 20 wherein the antihistamine is presented in a daily dose between 140 and 1120 μg/day.

24. The method according to claim 23 wherein the antihistamine is presented in a daily dose between 280 and 560 μg/day.

25. The method according to claim 20 wherein the corticosteroid is presented in a daily dose between 100 and 800 μg/day.

26. The method according to claim 25 wherein the corticosteroid is presented in a daily dose between 200 and 400 μg/day.

27. The method according to claim 20 wherein the PDE4 inhibitor is presented in a daily dose between 200 and 2000 μg/day.

28. The method according to claim 27 wherein the PDE4 inhibitor is presented in a daily dose between 400 and 1000 μg/day.

29. A method of treating non allergic types of rhinitis in an individual in need thereof comprising administering to said individual an effective amount of the combination of claim 1.

30. The method according to claim 29, wherein the rhinitis is vasomotor rhinitis or rebound rhinitis.

31. A method of treating rhinorrhea associated with common cold in an individual in need thereof comprising administering to said individual an effective amount of the combination of claim 1.
